(19) United States
(12) Patent Application Publication
(10) Pub. No.: US 2026/0013931 A1
(43) Pub. Date: Jan. 15, 2026

(54) DEVICES AND METHODS FOR TREATMENT OF TISSUE WITH MANUALLY DRIVEN ELECTRODES

(71) Applicant: pollogen ltd., Tel Aviv (IL)

(72) Inventors: Amikam GERSHONOWITZ, Tel Aviv (IL); Eyal Ben Esti, Tel Aviv (IL); Mordechai Moti Elnekave, Tel Aviv (IL); Aviad Harhol, Tel Aviv (IL)

(21) Appl. No.: 19/338,614

(22) Filed: Sep. 24, 2025

Related U.S. Application Data
(63) Continuation of application No. PCT/IL2024/050975, filed on Oct. 2, 2024.
(60) Provisional application No. 63/542,336, filed on Oct. 4, 2023.

(51) Int. Cl. A61B 18/14; A61B 18/00; A61B 18/12

(52) U.S. Cl. CPC A61B 18/1477; A61B 2018/0047; A61B 2018/00577; A61B 2018/00589; A61B 2018/0072; A61B 2018/00738; A61B 2018/00755; A61B 2018/00761; A61B 2018/126; A61B 2018/1475

(57) ABSTRACT

A tissue treatment device and system comprising a first stage having at least one elongated electrode with a length extending distally from the first stage and configured for receiving an electrical current signal from an electrical current source and for being inserted into the tissue to deliver the electrical current signal to the tissue thereby applying a treatment to the tissue. A second stage located distally to the first stage at a distance being at least equal to the length of the at least one elongated electrode, the second stage configured for being placed on surface of the tissue. A movement mechanism configured to: enable proximal displacement of the second stage to reveal a specific length of the at least one elongated electrode, under application of an external proximal force on the second stage and returning the second stage to its default location covering the distal end of the at least one elongated electrode, once the proximal force ends.

### RELATED APPLICATIONS

This application is a continuation application of PCT Application No. PCT/IL2024/050975, filed Oct. 2, 2024, which is related to U.S. Provisional Application No. 63/542,336 filed Oct. 4, 2023, entitled “DEVICES AND METHODS FOR TREATMENT OF TISSUE WITH MANUALLY DRIVEN ELECTRODES,” to which application priority is hereby claimed and the contents of which are incorporated herein by reference in its entirety.

### TECHNOLOGICAL FIELD

The present disclosure is in the medical, aesthetic, field and relates specifically to devices and methods for treatment of tissue, such as the skin, for aesthetic and/or cosmetic purposes. More specifically, the present disclosure relates to devices and methods for treatment of tissue of a subject by ablation or coagulation.

### BACKGROUND

Various skin treatment techniques for skin rejuvenation are available. Some treatments involve damaging the skin, e.g. by ablation or coagulation, to promote tissue revival. Specifically, some treatments are fractional, a term used to describe a form of treatment that creates an array of relatively small, discrete treatment spots in the skin tissue and leaves sites of healthy and untreated tissue around the treatment spots. The treatment spots may be created by optical (e.g. laser), electromagnetic (e.g. radio frequency), sound (e.g. ultrasound) or other energies/modalities. At each treatment spot a micro-damage is created in the skin tissue. The micro-damage at the treatment spot triggers a natural healing response of the skin tissue. The intact healthy tissue surrounding the treatment spots provides a basis for healing the micro-damage.

Examples for fractional skin treatment can be found in WO2021234609A1 and WO2021234605A1 both assigned to the assignee of the present invention and incorporated by reference herein in their entirety.

### SUMMARY

The presently disclosed subject matter provides techniques for treatment of tissue by delivering a controlled amount of electrical energy to locally damage the tissue and trigger a healing process.

Delivery of the electrical energy is done at one or more predetermined depths in the tissue using one or more elongated electrodes (may also be referred to herein as needles, pins, microneedles or micro-pins). The elongated electrodes are configured for insertion into the tissue to the predetermined one or more depths while an electrical signal is controllably applied to the tissue through the elongated electrode(s).

In accordance with the present disclosure, the insertion of the elongated electrode(s) into the tissue is done manually, to simplify the treatment process and save on using more complicated systems such as electrical motors. Also, in accordance with the current disclosure, pulling the elongated electrodes out of the tissue is obtained in a seamless and automatic way, as further described herein.

In some embodiments, the presently disclosed subject matter allows the user to determine the insertion depth of the elongated electrode(s) during a treatment. The devices disclosed are configured with mechanisms that enable setting maximal insertion depth, achieved with a repeatable manual activation of the device. At the same time, when not in use on the patient's body, the mechanisms ensure that the tip(s) of the elongated electrode(s) are concealed safely so as to prevent patient/operator injury and increase the safety.

In one aspect to the current disclosed invention there is at issue treatment device comprising: a first stage comprising at least one elongated electrode with a length extending distally from the first stage. The at least one elongated electrode configured to: receive an electrical current signal from an electrical current source; and be inserted into the tissue to deliver the electrical current signal to the tissue thereby applying a treatment to the tissue; a second stage located distally to the first stage at a distance being at least equal to the length of the at least one elongated electrode, the second stage configured to be placed on surface of the tissue. Also comprising a movement mechanism configured to: enable proximal displacement of the second stage to reveal a predetermined length of the at least one elongated electrode, under application of an external proximal force on the second stage; and to return the second stage to its default location covering the distal end of the at least one elongated electrode once the proximal force ends.

In another aspect there is a treatment tissue device wherein th movement mechanism is configured to enable the proximal displacement of the second stage to different fixed distances up to the predetermined length of the at least one elongated electrode to thereby reveal respective different fixed lengths of the at least one elongated electrode corresponding to different fixed penetration depths of the at least one elongated electrode into the tissue; and wherein the movement mechanism comprises a rotatable member configured when rotated to different angles to define the different fixed distances. The proximal force is achieved by placing the second stage on the surface of the tissue and pushing the first stage distally towards the surface of the tissue, the proximal force ends when an operator stops to push the first stage distally. The at least one elongated electrode is configured as a monopolar electrode, whereby the electrical current signal flows between the at least one electrode and a distant electrode positioned at a different part of the patient's body and having a current density much lower than a current density at the at least one elongated electrode.

In yet another aspect, the tissue treatment device comprises a plurality of the elongated electrode, the device being thereby configured to apply a fractional treatment to the tissue; and the plurality of the elongated electrode is configured as monopolar electrodes, the electrical current signal flows between each of the plurality of elongated electrodes and a distant electrode positioned at a different part of the patient's body and having a current density much lower than a current density at each of the plurality of elongated electrode.

In some aspects of the tissue treatment device, the plurality of said elongated electrode comprises a first group having a first polarity and a second group having a second polarity opposite to the first polarity, the plurality of elongated electrodes being thereby activated in a bipolar mode and the electrical current signal flows between the first and second electrode groups. The second stage forms a flat electrode having a second polarity opposite a first polarity of the at least one elongated electrode and having a current density much lower than a current density at the at least one elongated electrode, the electrical current signal thereby flows between the at least one elongated electrode and the flat electrode. The tissue treatment device comprises a plurality of said elongated electrode, the device being thereby configured to apply a fractional treatment to the tissue. The tissue treatment device comprising a proximal part and a distal part, the proximal part functioning as a handle to hold and manipulate the device, the distal part housing the first stage, the at least one elongated electrode, the second stage and the movement mechanism, the distal part is attachable to the proximal part in a way such that the first stage is spatially fixed with respect to a distal end of the proximal part. The tissue treatment device wherein the distal part is configured to be disposable; the proximal part comprises an electrical current source electrically couplable to the at least one elongated electrode; and the at least one elongated electrode is electrically insulated along its external surface apart from selected one or more electrically conductive portions thereof.

In an aspect, the tissue treatment device further comprises an electrical current source and a controller configured to activate the electrical current source to generate the electrical current signal, wherein said electrical current signal is one of: a direct current (DC) or an alternating current (AC); and the alternating current is in a radiofrequency (RF) range. The tissue treatment device wherein said electric current signal applies one or more of the following tissue treatments near the at least one elongated electrode: local tissue ablation or local tissue coagulation.

In another aspect, the tissue treatment device wherein the device further comprises a controller that is configured to dynamically and in real time determine parameters of the electrical current signal based on partial length of the at least one elongated electrode extending distally from the second stage, wherein the controller is configured to activate or determine parameters of the electric current signal based on at least one of the following: confirmation of device is placed adjacent to the tissue surface; depth of the at least one elongated electrode penetration in the tissue; or a penetration depth of the movement mechanism. The tissue treatment device wherein the depth of the at least one elongated electrode penetration in the tissue is determined by the controller being configured to: calculate impedance employing electrical characteristics of the at least one elongated electrode; and determine depth with a look-up table of impedance values as function of depth. The tissue treatment device, wherein the device further comprises sensors to sense at least one of the following: if device is placed adjacent to the tissue surface; a depth of the at least one elongated electrode penetration in the tissue; or a penetration depth of the movement mechanism.

In some aspects of the current disclosure there is a method for tissue treatment comprising: providing a first stage comprising at least one elongated electrode with a length extending distally from the first stage, a second stage located distally to the first stage at a distance being at least equal to the length of the at least one elongated electrode, a movement mechanism, and an electrical current source; placing the second stage on a surface of tissue; inserting the at least one elongated electrode into the tissue by applying a proximal displacement force of the second stage with an external proximal force on the second stage to reveal a predetermined length of the at least one elongated electrode; delivering an electrical current signal, received from the electrical current source, to the tissue thereby applying a treatment to the tissue; returning the second stage to its default location covering a distal end of the at least one elongated electrode once the proximal force ends. The movement mechanism further comprises a rotatable member configured to determine a fixed distance of the at least one electrode insertion into the tissue and the method further comprises rotating the rotatable member to an angle that determines the fixed distance of the at least one electrode.

In an aspect, the method further comprising applying a fractional treatment to the tissue, through a plurality of said elongated electrode; configuring the plurality of elongated electrode as bipolar electrodes, comprising a first group of the plurality of elongated electrode having a first polarity and a second group having a second polarity opposite to the first polarity, the electrical signal thereby flowing between the first and second electrode groups; or configuring the electrical current signal to flow between the at least one elongated electrode and a flat electrode formed by the second stage, whereby the flat electrode has a second polarity opposite of a first polarity of the at least one elongated electrode. The at least one elongated electrode is electrically insulated along its external surface apart from selected one or more electrically conductive portions thereof. The method further comprises activating an electrical current source of the device to generate the electrical current signal. The electrical current signal is one of: a direct current (DC) or an alternating current (AC), and said alternating current is in the radiofrequency (RF) range.

In another aspect, there is a method further comprising determining, dynamically and in real time, by a controller of the device, parameters of the electrical current signal based on a partial length of the at least one elongated electrode extending distally from the second stage; or applying one or more of the following tissue treatments near the at least one elongated electrode: local tissue ablation and local tissue coagulation. The method further comprises: activating or determining, by the controller, parameters of the electric current signal based on at least one of the following: confirmation of device is placed adjacent to the tissue surface; depth of the at least one elongated electrode penetration in the tissue; or a penetration depth of the movement mechanism. The method further comprises: calculating impedance, by the controller, employing electrical characteristics of the at least one elongated electrode; comparing, by the controller, calculated impedance to a look-up table of impedance values as function of depth; and determining, by the controller, the depth of the at least one elongated electrode penetration in the tissue.

In some aspects, the method further comprises: providing sensors; sensing information sensed of at least one of the following; (i) if device is placed adjacent to the tissue surface, (ii) a depth of the at least one elongated electrode penetration in the tissue, or (iii) a penetration depth of the movement mechanism; and transmitting, by the sensors to the controller, the information sensed.

In a final aspect, there is a tissue treatment device configured to be connected to a source of electric signal, comprising: a first stage comprising at least one elongated electrode with a length extending distally from the first stage, the at least one elongated electrode configured to be inserted into the tissue; a second stage located distally to the first stage at a distance being at least equal to the length of the at least one elongated electrode, the second stage configured to be placed on surface of the tissue; a movement mechanism with a stepped penetration depth limiter mechanism comprising a plurality of grooved steps at a bottom side of the first stage and a rotatable housing at a bottom side of the second stage with an internal radial structure. The movement mechanism configured to: enable a plurality of fixed different lengths of the at least one elongated electrode corresponding to proximal movement of the second stage; rotate the rotatable housing to a consecutive angle that aligns the internal radial structure with at least one of one of the plurality of grooved steps, thereby defining the length of the proximal displacement of the second stage; enable proximal displacement of the second stage to reveal a predetermined length of the at least one elongated electrode, under application of an external proximal force on the second stage; and return the second stage to its default location covering the distal end of the at least one elongated electrode once the proximal force ends.

### BRIEF DESCRIPTION OF THE DRAWINGS

FIGS. 1A-1B schematically illustrate a device for treatment of tissue, according to some embodiments.

FIG. 2 illustrates schematically illustrates a device configured according to some embodiments.

FIGS. 3A-3C illustrate a device configured according to some embodiments.

FIGS. 4A-4E illustrate a non-limiting example of a device configured in accordance some embodiments.

FIG. 5 illustrates a functional block diagram of a system for treatment of tissue, according to some embodiments.

FIG. 6 illustrates a flow chart of a method of tissue treatment, according to some embodiments.

### DETAILED DESCRIPTION OF EMBODIMENTS IN THE DRAWINGS

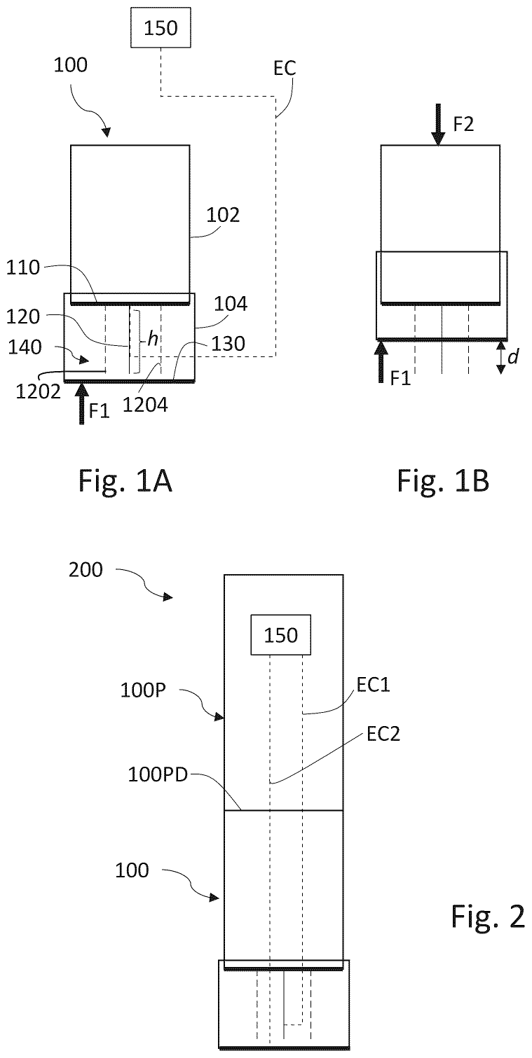

Reference is made to FIGS. 1A-1B, illustrating schematically a device 100 for treatment of tissue. The device 100 comprises a first stage 110 supporting at least one elongated electrode 120 having a length h extending distally from the first stage 110 and configured for receiving an electrical current signal EC (AC and/or DC) from an electrical current source 150 (that is not necessarily part of the device 100) and for being inserted into the tissue to deliver the electrical current signal to the tissue, thereby applying a treatment to the tissue.

The device 100 includes a second stage 130 located distally to the first stage 110 (i.e. farther from the operator of the device and closer to the treated tissue), at a distance being at least equal to the length h of the at least one elongated electrode 120. Therefore, the second stage 130 is configured to conceal the distal end of the at least one elongated electrode 120. The second stage 130 is configured for being placed on surface of the tissue to be treated.

A movement mechanism 140 is included in the device 100 as well and configured for a manual stamping action thereby; a) enabling proximal displacement of the second stage 130 to reveal a specific length d of the at least one elongated electrode 120 (FIG. 1B), under application of an external proximal force F1 on the second stage 130; and b) returning the second stage to its default location (FIG. 1A) covering the distal end of the at least one elongated electrode 120, once the proximal force F1 ends. The movement mechanism may enable, therefore, safe and fast tissue treatment to cover a large area of tissue, by sequentially treating adjacent tissue portions within a large tissue area. The movement mechanism enables fast and safe manual traversal of the device, whereby the manual stamping action is repeated over the large tissue area. In some embodiments, the stamping action is performed whereby the device is placed on a first tissue portion, and at least one elongated electrode is inserted by an operator of the movement mechanism into the first tissue portion and then pulled out, the device is moved to an adjacent second tissue portion and the at least one elongated electrode is again, by effect of the movement mechanism, inserted into the second tissue portion and pulled out, and so on until the entire area of treated tissue is traversed. As further described herein, the movement mechanism may enable the insertion of the at least one elongated electrode into the tissue by manually pushing the first stage distally against the second stage resting on the tissue.

The tissue treatment device 100 may be alternatively referred to as a microneedle electrode stamping device.

In some embodiments, the movement mechanism includes one or more elastic members, e.g. spring(s), that act in the opposite direction to the proximal force F1 acting on the second stage. In other words, the proximal force F1 acts against the one or more elastic members; However, as the proximal force F1 is larger in magnitude, the second stage 130 is moved proximally (upwardly) relative to the first stage, i.e. toward the first stage 110. When the proximal force F1 ends, the one or more elastic members are responsible for moving the second stage 130 distally (downwardly) back to the default position concealing the distal end of the at least one elongated electrode 120.

In some embodiments, the device includes a plurality of the at least one elongated electrode 120. In the non-limiting example shown, as illustrated in dashed lines, two additional elongated electrodes 1202 and 1204 are supported by the first stage 110. An array of the at least one elongated electrode 120 enables the device 100 to perform a fractional treatment of the tissue (i.e., several treatment spots spaced apart over an area of the tissue).

In some embodiments, as shown in this non-limiting example, the device 100 includes a first housing 102 and the first stage 110 is located at or forms the bottom side of the first housing 102. In some embodiments, as shown in this non-limiting example, the device 100 includes a second housing 104 and the second stage 130 is located at or forms the bottom side of the second housing 104. The second housing 104 may also, as shown in this example, have side walls extending, as a cover, proximally around the at least one elongated electrode to conceal it and further enhance safety of the device. In some embodiments, the proximal force F1 is achieved by placing the second stage 130 on the surface of the tissue and pushing the first stage 110, e.g. pushing the first housing 102, distally against the surface of the tissue, with a force F2 that is equal in magnitude and opposite in direction to the proximal force F1. This results in the relative movement of the second stage with respect to the first stage, such that the at least one elongated electrode projects distally and penetrates the tissue due to the mechanical forces combined with the tissue ablation or coagulation caused by the electrical current signal applied to the electrode(s). However, it is understood that there is another force acting distally by the movement mechanism (not shown) to return the second stage distally once the proximal force F1 ends. The proximal force F1 ends when an operator stops pushing the first stage distally; then the at least one electrode is pulled out by the movement mechanism out of the tissue and back to its proximal position with respect to the second stage.

In some embodiments, the at least one elongated electrode is configured as a monopolar electrode. The electrical current signal flows between the at least one electrode and a distant electrode positioned at a different part of the patient's body, such as the lower back or at an extremity different than or far from the treatment region. The distant electrode basically has a larger area contacting the body and therefore has a current density much lower than a current density at the at least one elongated electrode.

When a plurality of elongated electrodes is provided, the elongated electrodes may be activated in a monopolar or a bipolar mode. In the monopolar mode, all of the elongated electrodes will have the same polarity opposite a distant electrode's polarity. In the bipolar mode, the elongated electrodes may be activated as a first group (at least one elongated electrode) having a first polarity and a second group (at least one elongated electrode) having a second polarity opposite to the first polarity, the electrical current signal flows between the first and second electrode groups.

In some embodiments, there are three or more groups of elongated electrodes having a like number of polarities; this configuration is also known as a multipolar mode.

In some embodiments, the device is configured to be switched between two or more of monopolar mode, bipolar mode, and multipolar mode. The device may be configured for making a mode switch either only between treatments or even during a treatment. In some embodiments, more than one of the modes are operable simultaneously.

In some embodiments, the at least one elongated electrode has a pointed distal end facilitating its penetration into the tissue. In some embodiments, the at least one elongated electrode has a flat, blunt, distal end, that does not penetrate the tissue under reasonable force or without activation of an ablating electrical current signal.

In some embodiments, the second stage 130 forms or supports a flat electrode having a second polarity opposite a first polarity of the at least one elongated electrode. The flat electrode, as having a much larger contact area than the elongated electrode(s), has a current density much lower than a current density at the elongated electrode(s). The electrical current signal thereby flows between the at least one elongated electrode and the flat electrode that are in close proximity to each other.

In some embodiments, the at least one elongated electrode has a length of between 1-9 mm. In some embodiments, the at least one elongated electrode is electrically insulated along its external surface apart from selected one or more electrically conductive portions thereof. In one example, the at least one elongated electrode is insulated along its whole external surface apart from a distal portion of the elongated electrode. In one example, the conductive distal portion consists of the bottom side or face of the elongated electrode. It should be understood that when the device includes a plurality of elongated electrodes, they can have similar or different properties of what is described herein. For example, they may have similar or different lengths, similar or different transverse cross-sections, similar or different insulated or conductive configurations, similar or different supplied electrical current signals (magnitude, polarity, frequency . . . ), and so on. The selected configuration may be affected by various factors such as the tissue type, treatment site, desired treatment result, etc.

In some embodiments, the electrical current signal is an alternating current (AC). In some embodiments, the AC is in the range of radiofrequency (RF). In some embodiments, the electrical current signal is selected to cause a controlled tissue heating, ablation and/or coagulation.

Reference is made to FIG. 2 illustrating a device 200 configured according to non-limiting embodiments of the present invention. The device 200 may include a proximal part 100P and a distal part constituted by the device 100 described in FIGS. 1A-1B. The proximal and distal parts are attachable to each other, e.g. by known means in the art. The device 100 may be attachable to the proximal part 100P in a way such that the first stage is spatially fixed with respect to a distal end 100PD of the proximal part. The proximal part 100P functions as a handle to hold and manipulate the device 200. The distal part 100, as described above, houses the first stage, the at least one elongated electrode, the second stage and the movement mechanism. This configuration of the device 200 enables the device 100 to be disposable thereby enhancing the safety and simplicity by providing a sterile, single-use device.

In the described non-limiting example, the electrical current source 150 is housed within the proximal part 100P. The electrical current source 150 is electrically couplable to the at least one elongated electrode and to the second stage that forms a flat electrode as described above, as illustrated by the electrical currents EC1 and EC2. It should be understood that the electrical coupling may be achieved by different means including wired and/or wireless connections. In some embodiments, the device (100, 200) includes a controller (not shown) that enables control and activation of the electrical current source.

In this disclosure, a controller (in the singular) is understood to mean one or more controllers. The controller is in wired or wireless connection to one or more non-transitory computer-readable media that may comprise any combination of volatile memory, non-volatile memory, and storage devices. The controller may be connected wired or wirelessly to one or more I/O device interfaces that may allow for the connection of various I/O devices such as (keyboards, displays. mouse devices, pen input, etc. (not shown).

In some embodiments, the controller also enables receiving, from sensors or similar inputs, additional information, or parameters to analyze and effect the control and activation of the device. By way of specific example, if the controller receives a request for electrical current signal to be applied from the operator before the controller receives information that the at least one elongated electrode penetrates the tissue, the controller will delay the activation of the signal until such time that the at least one elongated electrode penetrates the skin.

In some embodiments, the controller is configured to dynamically and in real time control the electrical signal application based on the proximal movement of the second stage (that reveals a specific length of the elongated electrode(s) in the distal direction). For example, a first electrical signal is activated with a first predetermined proximal distance or distance range of the second stage, a second electrical signal is activated with a consecutive second predetermined proximal distance or distance range of the second stage, and so on.

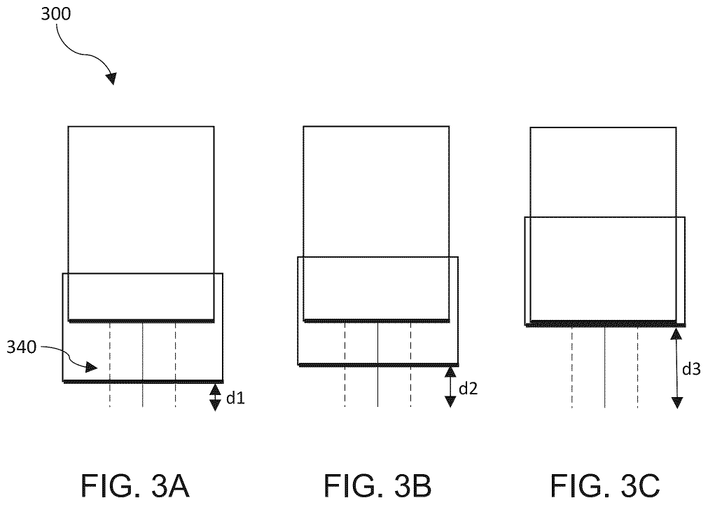

Reference is made to FIGS. 3A-3C illustrating a device 300 configured according to non-limiting embodiments of the present invention. The device 300 is similar to the device 100 described above with differences relating to additional capabilities of the movement mechanism 340. In this non-limiting example, the movement mechanism 340 is configured and operable to enable the proximal displacement of the second stage to different fixed distances up to the predetermined length of the at least one elongated electrode. By this, different fixed lengths of the at least one elongated electrode, corresponding to different fixed penetration depths into the tissue, are revealed distally to the moved up second stage. As shown in this non-limiting example, the movement mechanism is configured to enable three penetration depths d1, d2 and d3, corresponding to different revealed portions of the elongated electrodes.

This configuration enables selectively treating the tissue at different depths with the same device. For example, it might be desired to treat one tissue area with depth d1 and a second tissue area with depth d3. In another example, it might be desired to treat same tissue area at multiple vertical depths. This capability also enables random treatment pattern both horizontally and vertically, which may increase the treatment efficiency.

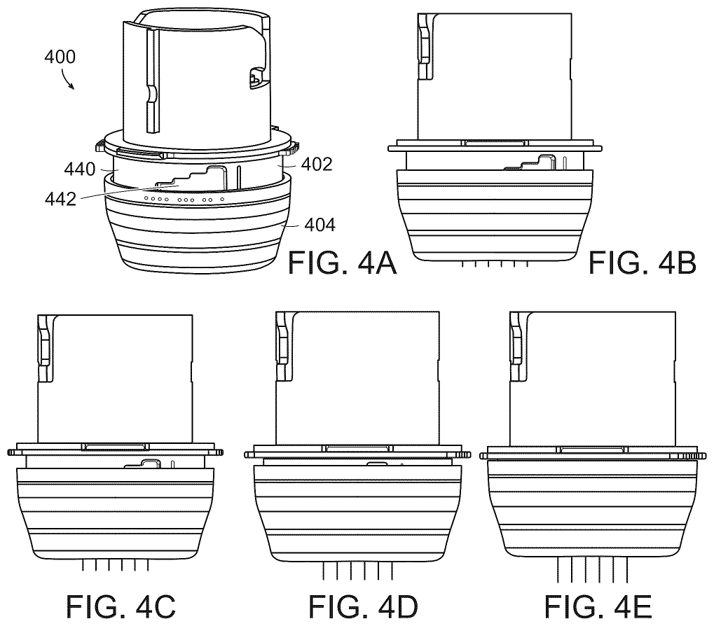

Reference is made to FIGS. 4A-4E showing a non-limiting example of a device 400 configured in accordance with the present invention. In this example, the device 400 includes a movement mechanism 440 configured to enable multiple penetration depths as described above in FIGS. 3A-3C. The movement mechanism 440 includes a stepped penetration depth definer/limiter mechanism 442 responsible to enable four fixed different projecting lengths/portions of the elongated electrodes corresponding to proximally moving up the second stage. While four fixed different lengths/portions are shown, it is to be understood that less than or more than four may be provided.

In some embodiments, the device 400 is a smart device or tip and employs a controller (not shown) with information gathered to inform treatment. In some embodiments, the controller receives from a sensor or the like an indication of which step of the stepped penetration depth the definer/limiter is set. In some embodiments, the controller receives information regarding the depth of the skin the needle has penetrated based on known methods such as calculating the impedance employing electrical characteristics of the electrodes. In some embodiments, the controller receives information to enable the recognition of skin in electrical contact with the needles, this may be calculated based on impedance employing the electrical characteristics of the electrodes and the tissue at different depths. In some embodiments, the impedance of the tissue is estimated based on depth into the tissue. For example, a look-up table of impedance values as function of depth may be utilized by the controller to adapt the electrical current signal, such as a RF signal, to the specific depth. In some embodiments, the controller of the tissue treatment device or system calculates impedance employing electrical characteristics of the elongated electrodes; and determines depth with the look-up table of impedance values as function of depth.

In some embodiments, the impedance of the tissue is estimated based on the tissue type. In some embodiments, the impedance is estimated based on combination of depth and tissue type. In some embodiments, there is a counter module in the device 400 that records how many times the device has performed the stamping action.

As shown in this non-limiting example, the stepped penetration depth definer/limiter mechanism 442 includes a groove 442G of four steps formed in external surface of the housing 402 supports at the bottom side the first stage and the elongated electrodes (similar to housing 102 above), and a rotatable second housing 404 supports at the bottom side the second stage (similar to housing 104 above). The rotatable second housing 404 has an internal radial structure such as a pin (not shown) that when aligned with one of the steps allows proximal displacement of the second housing 404 until the radial structure reaches the step, i.e. the proximal displacement is governed by the height of that step. Rotating the second housing to consecutive angles enables aligning the radial structure with one of the four steps, thereby defining the length of the proximal displacement of the second housing, as shown in FIGS. 4B-4E. In this specific example, the revealed lengths of the elongated electrodes are 1, 2, 3 and 4 mm in the FIGS. 4B, 4C, 4D and 4E respectively.

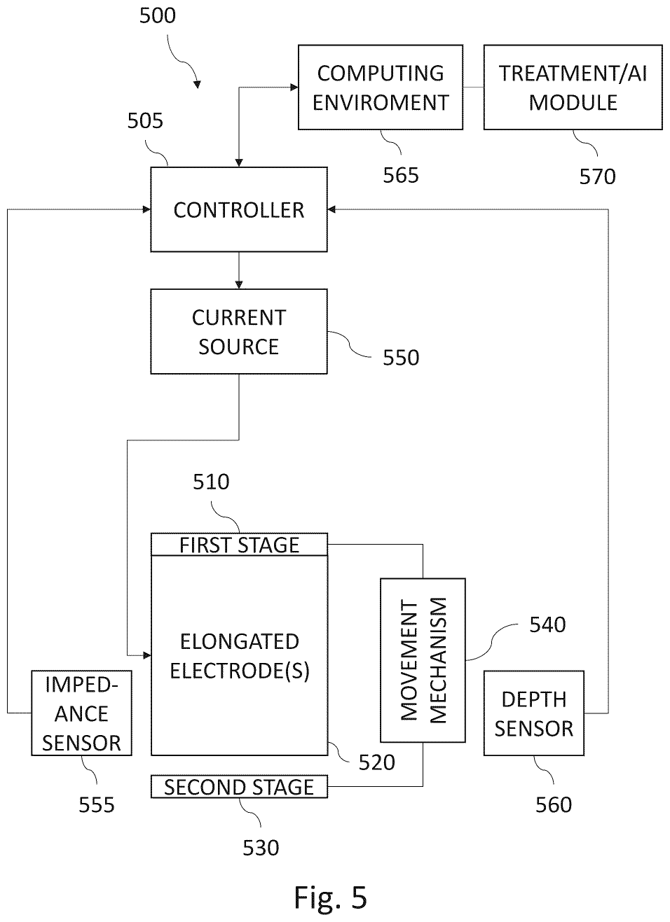

Reference is now made to FIG. 5, illustrating a functional block diagram of a system 500 for treatment of tissue with manually driven electrodes, according to some embodiments of the disclosure.

The system 500 may comprise a microneedle electrode stamping device, as further described herein. The stamping device may contain a first stage 510 in rigid connection with one or more elongated electrodes 520; a second stage 530, configured to be in contact with an external area of tissue; and a movement mechanism 540. The movement mechanism may be configured, when a force is applied to the first stage 510, to enable passage of the elongated electrodes 520 through holes of the second stage 530 and to pierce the tissue, reaching a predetermined distance through the tissue when the first stage 510 is pushed all the way. When the force is released, the elongated electrodes 520 retract from the tissue and return to their original position, i.e. non-protruding from the second stage 530.

The movement mechanism 540 may be further configured to adjust the predetermined distance, as further described herein.

The system 500 may further comprise a controller 505 and a current source 550. The controller 505 signals the current source 550 to apply a current through the elongated electrode(s) 520 and to stop applying the current. The controller 505 may further control the parameters of the current applied by the current source 550, such as one or more of current level, carrier wave frequency, pulse width, interphase interval, and repetition rate. It is understood that the controller 505 and the current source 550 may each be either in the housing of the device 500 or outside of and electrically connected to the device 500. It is also understood that features of the device as described in reference to FIG. 5 may also be applied to the device as described in reference to other figures.

In some embodiments, the controller instructs the device or system to supply from the current source a pulse-width modulation (PWM) of a carrier wave signal. The carrier wave may be sinusoidal, rectangular, or any other periodic wave. The carrier frequency may be in the range of 300 kHz to 2 Mhz. The carrier signal strength (RMS during the ON time of pulses) may be in a range of 5 V to 150 V. The pulse duration of the carrier wave envelope may be in a range from 300 ms to 2000 ms. The pulse repetition rate may be in the range from 10 Hz to 10 KHz. In some embodiments, the controller instructs the device or system to deliver a penetration pulse from the current source. In some embodiments, this penetration pulse has a maximum amplitude between about 75 root mean square voltage (VRMS) to 150 VRMS; and a penetration pulse duration between about 2 ms to 20 ms.

In some embodiments of the system 500, the controller 505 receives the setting of the stepped penetration depth of the definer/limiter (e.g., by a rotary switch to encode the angular position of the setting), and the controller 505 may adjust the applied current parameters according to the distance setting.

The system 500 may comprise an impedance sensor 555, in communicative connection with the controller 505. Based on the impedance, the controller 505 can stop the supply of current from the current source 550 when there is poor electrical contact of the elongated electrodes 520 and/or the second stage 530 with the tissue.

The system 500 may comprise a displacement sensor 560, in communicative connection with the controller 550. The displacement sensor 560 measures the present distance at which the elongated electrodes 520 are displaced. The displacement sensor 560 may comprise, for example, a linear encoder. The impedance sensor 555 may be used to function as a depth sensor 560 (either alone or in a multi-sensor configuration with a linear encoder), whereby the controller 550 may compute the penetration depth of the elongated electrodes 520 based on the impedance. The displacement sensor 560 enables the controller 505 to adjust the current parameters as a function of the penetration depth(s) of the elongated electrodes 520 into the tissue.

One or more other sensors (not shown) may be in communicative connection with the controller 505. For example, a temperature sensor, camera, heart rate sensor, blood pressure sensor, electrophysiological sensor(s) (e.g., EKG, ECG), and more. Readings from such sensors may help the controller 505 to determine adjustments to the parameters for a best course of treatment.

In some embodiments, the system 500 comprises a computing environment 565 (e.g., a single computer, a network, or a cloud environment). An end node such as a PC, tablet, or smartphone may be in communicative connection with the controller 505. The computing environment may receive sensor information from the controller 505 such as present impedance, displacement setting, present displacement, and other sensor readings acquired during treatment.

In some embodiments, the controller 505 alone may serve as the computing environment 565 with the treatment module 570; i.e., the system 500 is a stand-alone device including controller 505.

In some embodiments, an operator of the system 500 may manually adjust treatment parameters via a user interface of the computing environment 565.

The computing environment 565 may comprise or be in communicative connection with a treatment module 570. The treatment module 570 may contain a database of past treatments that enable the treatment module 570 to determine parameters for a best course of treatment, as a function of patient medical data and the desired outcome of the treatment. The treatment module 570 may receive sensor present readings acquired during treatment. Based on the sensor readings, the treatment module 570 may revise the course of treatment in real time, during the treatment.

In some embodiments, the treatment module has AI functionality, enabling the treatment module to learn the parameters for the best course of treatment, given fixed data such as medical data about the patient and the desired treatment outcome. Real-time adjustments of parameters for best course of treatment during the treatment may also be learned given the fixed data and the sensor readings.

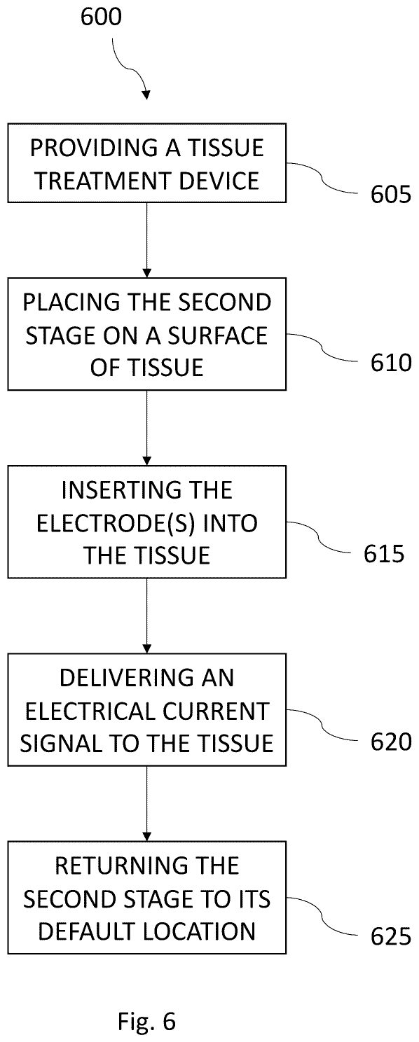

Reference is now made to FIG. 6, a flow chart for a method of tissue treatment 600, according to some embodiments.

The method 600 may comprise providing a tissue treatment device 605: a first stage comprising at least one elongated electrode with a length extending distally from the first stage, a second stage located distally to the first stage at a distance being at least equal to the length of the at least one elongated electrode, a movement mechanism, and an electrical current source.

The method 600 may comprise placing the second stage on a surface of tissue 610.

The method 600 may comprise inserting the at least one elongated electrode into the tissue 615 by applying a proximal displacement force of the second stage with an external proximal force on the second stage to reveal a predetermined length of the at least one elongated electrode.

The method 600 may comprise delivering an electrical current signal to the tissue 620, generated by the electrical current source through the at least one elongated electrode, thereby applying a treatment to the tissue.

The method 600 may comprise returning the second stage to its default location, covering a distal end of the at least one elongated electrode, once the proximal force ends 625.

As used herein, the term “dynamically” and its logical and/or linguistic relatives and/or derivatives, mean that actions or processes occur in real-time and/or are capable of adjusting behavior based on varying conditions or inputs. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

1. A tissue treatment device configured to be connected to a source of electric signal, comprising:
a first stage comprising at least one elongated electrode with a length extending distally from the first stage, the at least one elongated electrode having a distal end and being configured to be inserted into the tissue;
a second stage comprising an apertured flat electrode located distally of the first stage at a distance being at least equal to the length of the at least one elongated electrode, the second stage configured to be placed on surface of the tissue, wherein the apertured flat electrode is configured to have a polarity opposite to a polarity of the at least one elongated electrode when configured to be connected to the source of electrical signal;
a movement mechanism with a stepped penetration depth limiter mechanism comprising a plurality of grooved steps at a bottom side of the first stage and a rotatable housing at a bottom side of the second stage with an internal radial structure;
the movement mechanism configured to:
enable a plurality of fixed different lengths of the at least one elongated electrode to penetrate apertured in the apertured flat electrode corresponding to proximal movement of the second stage;
rotate the rotatable housing to a consecutive angle that aligns the internal radial structure with at least one of one of the plurality of grooved steps, thereby defining the length of the proximal displacement of the second stage;
enable upward displacement of the second stage towards the first stage, to reveal a predetermined length of the at least one elongated electrode through the apertures in the apertured flat electrode, under application of an external force on the second stage; and
return the second stage to its default location upon removal of the external force.

2. A tissue treatment device comprising:
a first stage comprising at least one elongated electrode with a length extending distally from the first stage, the at least one elongated electrode configured to:
receive an electrical current signal from an electrical current source; and
be inserted into the tissue to deliver the electrical current signal to the tissue thereby applying a treatment to the tissue;
a second stage located distally to the first stage at a distance being at least equal to the length of the at least one elongated electrode, the second stage configured to be placed on a surface of the tissue, wherein the second stage comprises an apertured flat electrode having an opposite polarity to a polarity of the at least one elongated electrode, whereby the electrical current signal flows between the at least one elongated electrode and the second stage; and
a movement mechanism configured to:
enable upward displacement of the second stage towards the first stage to reveal a predetermined length of the at least one elongated electrode, under application of an external force on the second stage; and
return the second stage to its default location upon removal of the external force ends.

3. The tissue treatment device of claim 2, wherein said movement mechanism is configured to enable the upward displacement of the second stage to different fixed distances up to the predetermined length of the at least one elongated electrode to thereby reveal respective different fixed lengths of the at least one elongated electrode corresponding to different fixed penetration depths of the at least one elongated electrode into the tissue.

4. The tissue treatment device of claim 2, wherein said movement mechanism comprises a rotatable member configured when rotated to different angles to define different revealed lengths of the at least one elongated electrode.

5. The tissue treatment device of claim 4, wherein the rotatable member is configured to enable multiple stepped penetration depths of the at least one elongated electrode.

6. The tissue treatment device of claim 2, wherein the movement mechanism comprises one or more elastic members configured to act against and in an opposite direction of said external force, wherein the one or more elastic members are configured to move the second stage distally back to the default location covering the distal end of the at least one elongated electrode, when the external force ends.

7. The tissue treatment device of claim 2, wherein the second stage comprises at least one aperture adapted for passage of the at least one elongated electrode; wherein the movement mechanism is configured to enable the passage of the at least one elongated electrode through the at least one hole of the second stage to pierce the tissue, reaching a predetermined distance through the tissue.

8. The tissue treatment device of claim 2, wherein the at least one elongated electrode comprising a plurality of elongated electrodes, whereby the device being configured to apply a fractional treatment to the tissue.

9. The tissue treatment device of claim 8, wherein a distance between each of the at least one elongated electrode and surrounding portions of the flat electrode is constant.

10. The tissue treatment device of claim 8, wherein said plurality of said elongated electrode comprises a first group having a first polarity and a second group having a second polarity opposite to the first polarity, the plurality of elongated electrodes further being thereby activated in a bipolar mode and the electrical current signal further flows between the first and second electrode groups.

11. The tissue treatment device of claim 2, wherein said second stage having a current density much lower than a current density at the at least one elongated electrode, whereby enabling ablation of the tissue contacting the at least one elongated electrode.

12. The tissue treatment device of claim 2, wherein the at least one elongated electrode is electrically insulated along its external surface apart from selected one or more electrically conductive portions thereof.

13. The tissue treatment device of claim 2, comprising a proximal part and a distal part, the proximal part functioning as a handle to hold and manipulate the device, the distal part housing the first stage, the at least one elongated electrode, the second stage and the movement mechanism, the distal part is attachable to the proximal part in a way such that the first stage is spatially fixed with respect to a distal end of the proximal part.

14. The tissue treatment device of claim 13, wherein said distal part is configured to be disposable.

15. The tissue treatment device of claim 13, wherein the proximal part comprises the electrical current source being electrically couplable to the at least one elongated electrode.

16. The tissue treatment device of claim 13, wherein said electrical current signal is one of: a direct current (DC) or an alternating current (AC).

17. The tissue treatment device of claim 16, wherein said alternating current is in a radiofrequency (RF) range.

18. The tissue treatment device of claim 2, comprising the electrical current source and a controller configured to activate the electrical current source to generate the electrical current signal.

19. The tissue treatment device of claim 18, wherein the controller is further configured to dynamically and in real time determine parameters of the electrical current signal based on movement of the second stage, whereby different electrical signals are activated with different distance ranges of the second stage.

20. The tissue treatment device of claim 18, wherein the controller is further configured to determine parameters of the electrical current signal, wherein the parameters of the electric current signal comprise a pulse intensity and pulse duration.

21. The tissue treatment device of claim 20, wherein the controller is configured to activate the electrical current source to deliver a penetration signal followed by a treatment signal, the penetration signal having a higher intensity and a lower duration comparing to the treatment signal, wherein the penetration signal is configured to enable insertion of the at least one elongated electrode into the tissue by ablation of the tissue.

22. The tissue treatment device of claim 18, wherein the controller is further configured to activate or determine parameters of the electric current signal based on at least one of the following:
confirmation of the tissue treatment device being placed adjacent to the tissue surface;
depth of the at least one elongated electrode penetration in the tissue; or
a penetration depth enabled by the movement mechanism.

23. The tissue treatment device of claim 22, wherein the depth of the at least one elongated electrode penetration in the tissue is determined by the controller being configured to:
calculate impedance employing electrical characteristics of the at least one elongated electrode; and
determine depth with a look-up table of impedance values as function of depth.

24. The tissue treatment device of claim 23, wherein the device further comprises sensors to sense at least one of the following:
if device is placed adjacent to the tissue surface;
a depth of the at least one elongated electrode penetration in the tissue; or
a penetration depth enabled by the movement mechanism.

25. The tissue treatment device of claim 2, wherein said electric current signal applies one or more of the following tissue treatments near the at least one elongated electrode: local tissue ablation or local tissue coagulation.

26. The tissue treatment device of claim 2, wherein the at least one elongated electrode is configured to be inserted into the tissue by a combination of the external force and a tissue ablation caused by the electrical current signal, wherein in response to the external force causing projecting of the at least one elongated electrode to the tissue, the electrical current signal flowing between the at least one elongated electrode and the second stage being placed on the surface of the tissue causes the tissue ablation enabling the at least one elongated electrode to be inserted into the tissue.

27. The tissue treatment device of claim 2, wherein the at least one elongated electrode has a has a flat, blunt, distal end, that does not penetrate the tissue under a force lower than the external force or without activation of an ablating electrical current signal.

28. A method for tissue treatment comprising:
providing:
a first stage comprising at least one elongated electrode with a length extending distally from the first stage,
a second stage located distally to the first stage at a distance being at least equal to the length of the at least one elongated electrode, wherein the second stage comprises a flat electrode;
a movement mechanism, and
an electrical current source;
placing the second stage on a surface of tissue;
applying an external force on the second stage to enable an upward displacement of the second stage towards the first stage, whereby revealing a predetermined length of the at least one elongated electrode onto the surface of the tissue;
inserting the at least one elongated electrode into the tissue;
delivering an electrical current signal, received from the electrical current source to the tissue thereby applying a treatment to the tissue, wherein said delivering the electrical current signal comprises:
delivering an electrical current signal having a polarity from the electrical current source to the at least one elongated electrode; and
providing an electrical current signal having an opposite to the polarity of the at least one elongated electrode, whereby enabling the electrical current signal to flow between the at least one elongated electrode and the second stage;
returning, by the movement mechanism, the second stage to its default location covering a distal end of the at least one elongated electrode once the proximal force ends.

29. The method of claim 28, wherein said inserting the at least one elongated electrode into the tissue is enabled by a combination of the external force applied on the second stage and an ablation of the tissue surface contacting the at least one elongated electrode cause by said delivering an electrical current signal.

30. The method of claim 28, wherein said movement mechanism is configured to enable the upward displacement of the second stage to different fixed distances up to the predetermined length of the at least one elongated electrode to thereby reveal respective different fixed lengths of the at least one elongated electrode corresponding to different fixed penetration depths of the at least one elongated electrode into the tissue.

31. The method of claim 30, the movement mechanism further comprises a rotatable member configured to define when rotated to different angles to define the different revealed lengths of the at least one elongated electrode, wherein the method further comprises:
rotating the rotatable member to an angle that defines a predetermined fixed distance of the at least one electrode.

32. The method of claim 31, wherein the rotatable member is configured to enable multiple stepped penetration depths of the at least one elongated electrode.

33. The method of claim 32, wherein the rotatable member is configured to enable at least four fixed different revealed lengths of the at least one elongated electrode corresponding to upward displacement of the second stage

34. The method of claim 28, wherein said applying the external force comprises placing the second stage on the surface of the tissue and manually pushing the first stage distally towards the surface of the tissue, the external force ends when said manually pushing the first stage distally stops.

35. The method of claim 34, wherein the movement mechanism comprises one or more elastic members configured to act against and in an opposite direction of said external force, wherein said returning comprises, moving, by the one or more elastic members, the second stage distally back to the default location covering the distal end of the at least one elongated electrode.

36. The method of claim 28, wherein the at least one elongated electrode comprises a plurality of elongated electrodes, wherein said applying the treatment to the tissue comprises applying a fractional treatment to the tissue.

37. The method of claim 36, wherein the plurality of the elongated electrode comprises a first group having a first polarity and a second group having a second polarity opposite to the first polarity, wherein said delivering the electrical current signal comprises activating the plurality of elongated electrodes further in a bipolar mode and whereby the electrical current signal further flows between the first and second electrode groups.

38. The method of claim 28, wherein the second stage having a current density much lower than a current density at the at least one elongated electrode, whereby enabling ablation of the tissue contacting the at least one elongated electrode.

39. The method of claim 28, wherein the at least one elongated electrode is electrically insulated along its external surface apart from selected one or more electrically conductive portions thereof.

40. The method of claim 28, further comprising activating an electrical current source of the device to generate the electrical current signal.

41. The method of claim 40, wherein the electrical current signal is one of: a direct current (DC) or an alternating current (AC).

42. The method of claim 41, wherein the alternating current is in a radiofrequency (RF) range.

43. The method of claim 28, further comprises:
providing a controller configured to activate the electrical current source to generate the electrical current signal.

44. The method of claim 43, further comprises:
determining, by the controller, dynamically and in real time, parameters of the electrical current signal based on movement of the second stage; and
activating different electrical signals with different distance ranges of the second stage.

45. The method of claim 44, further comprises:
automatically determining, by the controller, parameters of the electrical current signal, wherein the parameters of the electric current signal comprise a pulse intensity and pulse duration.

46. The method of claim 45, further comprises:
activating the electrical current source to deliver a penetration signal followed by a treatment signal, the penetration signal having a higher intensity and a lower duration comparing to the treatment signal, wherein a penetration signal is configured to enable insertion of the at least one elongated electrode into the tissue by ablation of the tissue.

47. The method of claim 43, further comprises determining or activating parameters of the electric current signal based on at least one of the following:
confirmation of the tissue treatment device being placed adjacent to the tissue surface;
depth of the at least one elongated electrode penetration in the tissue; or
a penetration depth enabled by the movement mechanism.

48. The method of claim 47, further comprises determining the depth of the at least one elongated electrode penetration in the tissue, wherein said determining comprises:
calculating impedance employing electrical characteristics of the at least one elongated electrode;
comparing, calculated impedance to a look-up table of impedance values as function of depth; and
determining the depth of the at least one elongated electrode penetration in the tissue.

49. The method of claim 48, further comprises:
providing sensors;
sensing using the sensors, at least one of the following:
if device is placed adjacent to the tissue surface;
a depth of the at least one elongated electrode penetration in the tissue; or
a penetration depth enabled by the movement mechanism; and
transmitting, by the sensors to the controller, the information sensed.

50. The method of claim 28, wherein said applying the treatment comprises applying one or more of the following tissue treatments near the at least one elongated electrode: local tissue ablation or local tissue coagulation.

51. The method of claim 28, wherein said inserting the at least one elongated electrode is performed by a combination of the external force and a tissue ablation caused by the electrical current signal, wherein in response to the external force causes projecting the at least one elongated electrode to the tissue, the electrical current signal flowing between the at least one elongated electrode and the second stage being placed on the surface of the tissue causes the tissue ablation enabling the at least one elongated electrode to be inserted into the tissue.

52. The method of claim 28, wherein the at least one elongated electrode has a has a flat, blunt, distal end, that does not penetrate the tissue under a force lower than the external force or without activation of an ablating electrical current signal.
